(19) United States
(12) Patent Application Publication
(10) Pub. No.: US 2025/0127448 A1
(43) Pub. Date: Apr. 24, 2025

(54) LEAD CONFIGURED TO DETECT STROKE METRICS

(71) Applicant: Covidien LP, Mansfield, MA (US)

(72) Inventors: Arun Kumar Sathiyamoorthy, Chennai (IN); Randal C. Schulhauser, Phoenix, AZ (US)

(21) Appl. No.: 18/919,912

(22) Filed: Oct. 18, 2024

Related U.S. Application Data
(60) Provisional application No. 63/591,922, filed on Oct. 20, 2023.

(51) Int. Cl. A61B 5/291; A61B 5/00

(52) U.S. Cl. CPC A61B 5/291; A61B 5/7282

(57) ABSTRACT

An example lead includes a plurality of electrodes configured to sense electrical signals from a patient; one or more light emitting diodes (LEDs); and one or more optical sensors configured to sense optical signals, wherein the LEDs, the optical sensors and the electrodes are respectively aligned in measuring pairs, the measuring pairs including: one of the LEDs or one of the optical sensors, and one of the plurality of electrodes.

### CROSS-REFERENCE TO RELATED APPLICATIONS

This application claims the benefit of and priority to U.S. Provisional Patent Application Ser. No. 63/591,922 filed Oct. 20, 2023, the entire disclosure of which is incorporated by reference herein.

### TECHNICAL FIELD

This disclosure is directed to medical devices and, more particularly, to detection of stroke, brain ischemia, perfusion pressure, and/or hypoxia events.

### BACKGROUND

Stroke is a serious medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis or embolism, or hemorrhage (e.g., a ruptured blood vessel). During a stroke, the blood supply to an area of a brain may be decreased, which can lead to dysfunction of the brain tissue in that area.

Stroke is the number two cause of death worldwide and the number one cause of disability. Speed to treatment is the critical factor in stroke treatment as 1.9 M neurons are lost per minute on average during stroke. Stroke diagnosis and time between event and therapy delivery are the primary barriers to improving therapy effectiveness. Stroke has 3 primary etiologies; i) ischemic stroke (representing approximately 65% of all strokes), ii) hemorrhagic stroke (representing approximately 10% of all strokes), and iii) cryptogenic strokes (includes TIA, representing approximately 25% of all strokes). In an ischemic stroke, a blood clot occludes blow flow in an artery within the brain. In a hemorrhagic stroke, a blood vessel bursts within the brain. Strokes can be considered as having neurogenic and/or cardiogenic origins.

A variety of approaches exist for treating patients undergoing a stroke. For example, a clinician may administer anticoagulants, such as warfarin, or may undertake intravascular interventions such as thrombectomy procedures to treat ischemic stroke. As another example, a clinician may administer antihypertensive drugs, such as beta blockers (e.g., Labetalol) and ACE-inhibitors (e.g., Enalapril) or may undertake intravascular interventions such as coil embolization to treat hemorrhagic stroke. Lastly, if stroke symptoms have resolved on their own with negative neurological work-up, a clinician may administer long-term cardiac monitoring (external or implantable) to determine potential cardiac origins of cryptogenic stroke.

### SUMMARY

In general, the disclosure is directed to devices, systems, and techniques for detecting a cranial health event, such as a stroke, via leads, including both electrodes and optical sensors, located on the head of a patient. While stroke is the example cranial health event discussed primarily herein, the devices, systems, and techniques may additionally or alternatively be applied in the same or a similar manner to detect cranial health events such as high perfusion pressure, such as intracranial pressure (ICP), brain ischemia, and/or hypoxia events.

The lead may comprise a plurality of electrodes configured to sense electrical signals from a patient and processing circuitry may generate electroencephalography (EEG) signal(s) based on the electrical signals. The lead may further comprise one or more light emitting diodes (LEDs) and optical sensors configured to sense optical signals. The LEDs, optical sensors and the electrodes may be respectively aligned in measuring pairs, the measuring pairs including one of the LEDs or one of the optical sensors and one of the electrodes. The lead may be configured for implantation on or near the head, e.g., subcutaneously, transcutaneously, endovascularly, or otherwise beneath the scalp and above the cranium.

The techniques of this disclosure may provide one or more advantages. For example, the use of one or more leads including electrodes, LEDs, and optical sensors may enable generation of EEG signals and determination of cerebral blood flow or tissue oxygenation, such as brain tissue, via signals sensed by the same lead(s), which is less invasive as a means for detecting stroke and other cranial health events than a drain. Less invasively determining cerebral blood flow or tissue oxygenation reduces chances of patient having an infection or other side-effects of invasive procedures. As another example, the signals sensed via leads configured as described herein may include fewer motion artifacts or other noise than signals sensed via cutaneous or wearable sensors.

In addition, generation of EEG signals and determination of cerebral blood flow or tissue oxygenation via signals sensed by the same lead(s) may enable simultaneous or substantially simultaneous sensing of such signals, which may lead to analysis of time and spatially correlated signals for detection/localization of a stroke or other cranial health events with greater specificity and sensitivity.

In this manner, the techniques of this disclosure may help generate stroke detection that is both less invasive and less prone to noise than some existing techniques, and with greater specificity and sensitivity, especially for high-risk patients, such as after an initial stroke event occurs.

In one example, this disclosure describes lead comprising: a plurality of electrodes configured to sense electrical signals from a patient; one or more light emitting diodes (LEDs); and one or more optical sensors configured to sense optical signals, wherein the LEDs, the optical sensors and the electrodes are respectively aligned in measuring pairs, the measuring pairs including: one of the LEDs or one of the optical sensors, and one of the plurality of electrodes.

In another example, this disclosure describes a system comprising: a memory; a lead comprising: a plurality of electrodes; a plurality of near-infrared (NIR) light emitting diodes (LED); and a plurality of optical sensors configured to sense optical signals; processing circuitry configured to: sense electrical signals from a patient during a period of time; and generate, based on the electrical signals, one or more electroencephalography (EEG) signals; sense, via at least one of the optical sensors and using at least one of the LEDs, optical signals during the period of time; determine, based on the optical signals, one or more of cerebral blood flow or blood oxygenation; generate a metric indicative of a status of the patient based on the one or more EEG signals and the one or more of cerebral blood flow or blood oxygenation; and store the metric in the memory.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A is a conceptual diagram of a lead configured to detect electrical signals and optical signals in accordance with examples of the present disclosure.

FIG. 1B is a conceptual diagram of a system including a wireless lead configured to detect electrical signals and optical signals in accordance with examples of the present disclosure.

FIG. 1C is a conceptual diagram of a system including a wired lead configured to detect electrical signals and optical signals in accordance with examples of the present disclosure.

FIG. 1D is a block diagram of an example configuration of the controller in accordance with examples of the present disclosure.

FIG. 2A depicts a top view of a head of a patient in conjunction with a plurality of example leads, and depicts example target areas at which leads may be positioned on the head in accordance with examples of the present disclosure.

FIGS. 2B and 2C depict respective side views of a head of a patient in conjunction with an example lead, and depict example target areas at which leads may be positioned on the head in accordance with examples of the present disclosure.

FIG. 2D depicts a front view of a head of a patient in conjunction with an example lead, and depicts an example target area at which the lead may be positioned on the head in accordance with examples of the present disclosure.

FIG. 3A is a conceptual diagram of a system to configured detect stroke in accordance with examples of the present disclosure.

FIG. 3B is a conceptual diagram of a system configured to detect stroke in accordance with examples of the present disclosure.

FIG. 3C is a diagram of the 10-20 map for electroencephalography (EEG) sensor measurements.

FIG. 4 is a block diagram of an example configuration of the external device of FIGS. 1B-1C, 3A-3B.

FIG. 5 is a block diagram illustrating an example system that includes an access point, a network, external computing devices, such as a server, and one or more other computing devices, which may be coupled to the lead(s) and/or IMD, the external device, and the processing circuitry of FIG. 1 via a network, in accordance with one or more techniques described herein.

FIG. 6 is a flow diagram of an example technique for determining a metric of a patient.

Many aspects of the present disclosure can be better understood with reference to the following drawings. The components in the drawings are not necessarily to scale. Instead, emphasis is placed on clearly illustrating the principles of the present technology.

### DETAILED DESCRIPTION

It can be difficult to determine whether a patient is suffering from a stroke or has suffered from a stroke. Current diagnostic techniques typically involve evaluating a patient for visible symptoms, such as paralysis or numbness of the face, arm, or leg, as well as difficultly walking, speaking, balancing, seeing, or understanding (e.g., the B.E.F.A.S.T visible stroke indication of Balance, Eyes, Face, Arm, Speech, Time to call for emergency help). However, these techniques may result in undiagnosed strokes, particularly more minor strokes that leave patients relatively functional upon cursory evaluation. Other diagnostic techniques may include evaluating imaging, such as a computerized tomography (CT) scan or magnetic resonance imagining (MRI), which usually needs to be performed in a medical office or hospital.

Even for relatively minor strokes, it is important to treat the patient as soon as possible because treatment outcomes for stroke patients are highly time-dependent. However, such treatments may be frequently underutilized and/or relatively ineffective due to the failure to timely identify whether a patient is undergoing or has recently undergone a stroke. This is a particular risk with more minor strokes that leave patients relatively functional upon cursory evaluation.

Patients that are at high-risk of suffering a stroke or high perfusion pressures, such as dangerously high ICPs, may include a patient who has already suffered a stroke, a patient undergoing a medical procedure, such as a surgery, and/or a patient with physiological information that indicate the patient is at high-risk of a stroke. For these high-risk patients, it may be more difficult and/or more important to identify a stroke, even a relatively minor stroke, or that high perfusion pressures are occurring as soon as possible so treatments may be quickly utilized to increase their effectiveness, which may lead to improved patient outcomes and/or reduced medical costs.

Accordingly, there is a need for improved devices, systems, and methods for detecting strokes or high perfusion pressures. This disclosure describes various systems, devices, and techniques for determining a metric, such as a stroke metric or a perfusion pressure metric, such as ICP, using one or more leads, such as subcutaneous leads. The leads include a plurality of electrodes configured to sense electrical signals from a patient that are used to generate EEG signal(s) based on the electrical signals. The leads further include one or more LEDs and one or more optical sensors configured to sense optical signals. The LEDs, optical sensors and the electrodes may be respectively aligned in measuring pairs, each of the measuring pairs including one of the LEDs or one of the optical sensors and one of the electrodes. The use of one or more leads including electrodes, LEDs, and optical sensors may enable the same lead(s) to sense signals so processing circuity may generate EEG signals and determine cerebral blood flow or tissue oxygenation, such as brain tissue oxygenation, via signals sensed by the same lead(s). The leads and systems, as described herein, may enable to less invasive determinations of patient parameters, such as cerebral blood flow or tissue oxygenation. The leads and systems, as described herein, may also enable greater specificity and sensitivity of determinations of stroke indications and/or determination of high perfusion pressures because the signals being used to determine of patient parameters, such as EEG signals and cerebral blood flow and/or tissue oxygenation, may be sensed simultaneously or substantially simultaneously.

Aspects of the technology described herein can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., such as via Bluetooth®)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g., a Bluetooth® network) on a propagated signal on a propagation medium (e.g., an electromagnetic wave(s), a sound wave) over a period of time, or may be provided on any analog or digital network (packet switched, circuit switched, or other scheme).

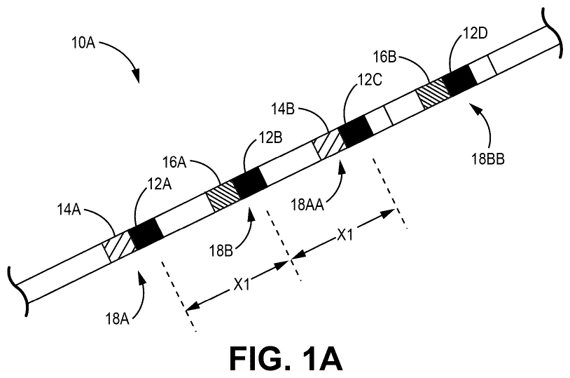

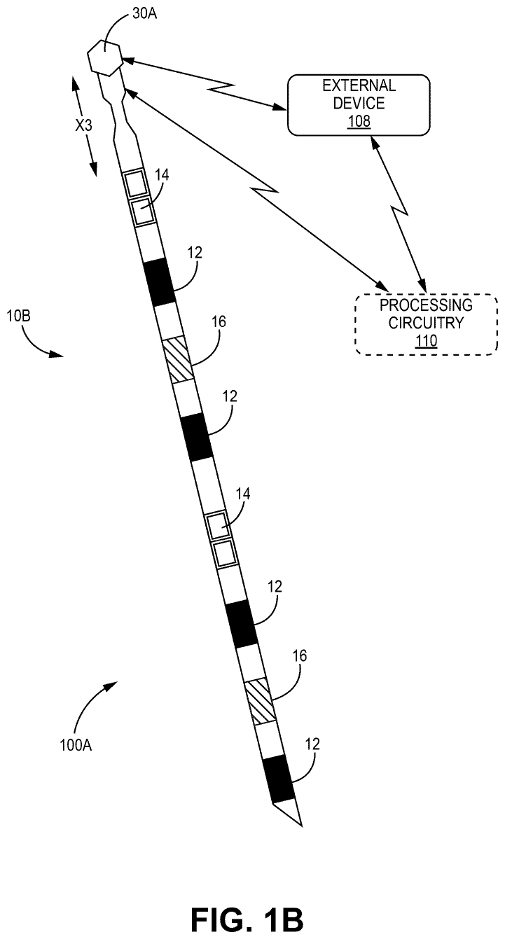

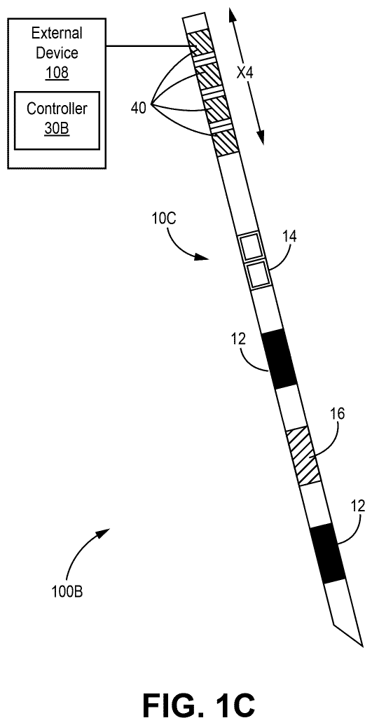

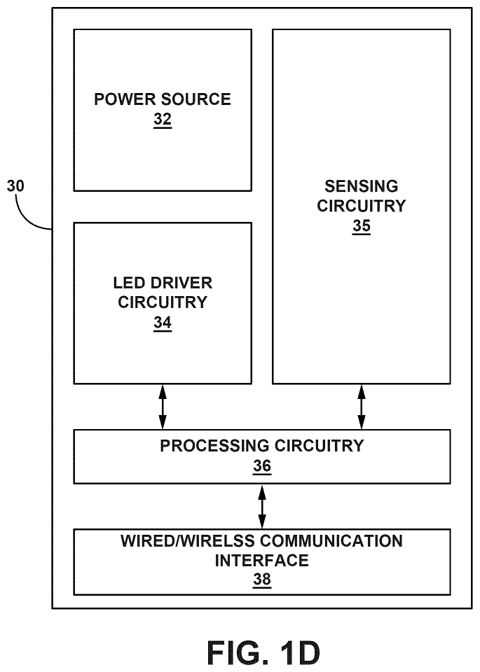

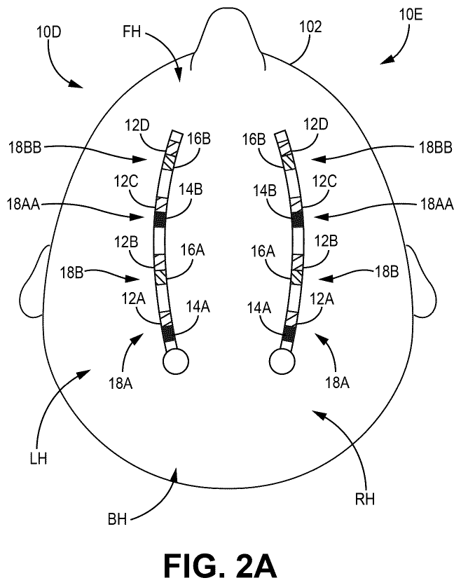

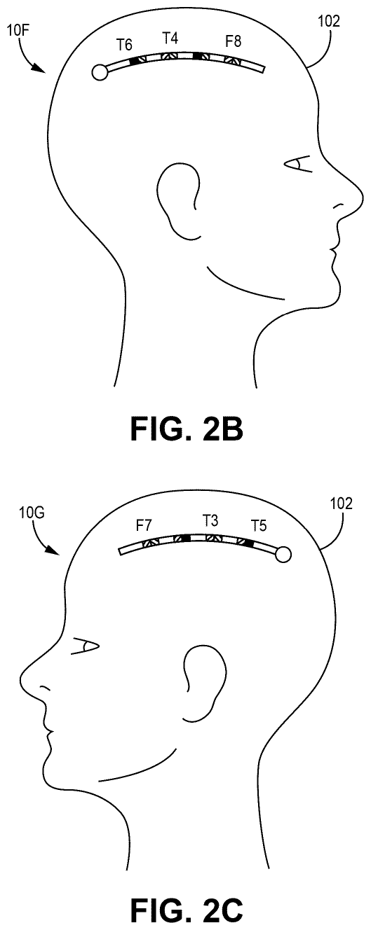

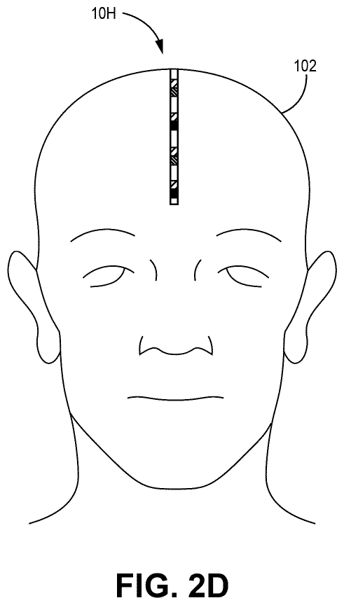

FIG. 1A is a conceptual diagram of a lead 10A configured to enable detection stroke or other cranial health events in a patient, such as a high-risk patient, in accordance with examples of the present disclosure. In some examples, lead 10A may include electrodes 12, light-emitting diodes (LED) 14 and optical sensors 16. In some examples, as shown in FIG. 1A, lead 10A may include electrode 12A, electrode 12B, electrode 12C, and electrode 12D (collectively electrodes 12). In some examples, as shown in FIG. 1A, lead 10A may include LED 14A and LED 14B (collectively LEDs 14). In some examples, one or more of LEDs 14 may be a near-infrared (NIR) LED. In some examples, as shown in FIG. 1A, lead 10A may include optical sensor 16A and optical sensor 16B (collectively optical sensors 16). In some examples, one or more of the optical sensors 16 may be a photodiode. While FIGS. 1A-2D may refer to a particular lead, such as lead 10A, 10D, 10E, etc., reference to lead 10 or leads 10 may refer to any lead(s) discussed herein. In some examples, one or more leads 10 may be implanted subcutaneously. In some examples, one or more leads 10 may be implanted subcutaneously or transcutaneously. In some cases, one or more leads 10 may be implanted in a subgaleal location of patient 102, such as between the skin on the scalp and the skull of a patient.

In some examples, the LEDs 14, the optical sensors 16 and the electrodes 12 may be respectively aligned in measuring pairs 18. In some examples, a measuring pair may include one of the LEDs 14 or one of the optical sensors 16 and one of the electrodes 12. For example, measuring pair 18A includes electrode 12A and LED 14A and measuring pair 18B includes electrode 12B and optical sensor 16A. In some examples, a measuring pair 18A that includes one LED 14 and one electrode 12 may be referred to as a first measuring pair 18A. In some examples, a measuring pair 18B that includes one optical sensor 16 and one electrode 12 may be referred to as a second measuring pair 18B. In some examples, as shown in FIG. 1A, lead 10A may include a plurality of first measuring pairs 18A and second measuring pairs 18B. In some examples, an electrode 12 may be adjacent to, such as directly adjacent to and/or axially adjacent to, an LED 14 or optical sensor 16 of a respective measuring pair 18. In some examples, such as shown in FIGS. 1B-1C, an electrode 12 may be separated by a distance to an LED 14 or optical sensor 16 of a respective measuring pair 18.

In some examples, as shown in FIG. 1A, first measuring pairs 18A and the second measuring pairs 18B are aligned alternatively in an axial direction of lead 10A. Each of the measuring pairs 18 may be respectively separated from an adjacent measuring pair of the measuring pairs by a predetermined distance, such as X1 or X2, as shown in FIG. 1A. For example, as shown as an example in FIG. 1A, measuring pair 18A is separated from measuring pair 18B, in the axial direction of lead 10A, by distance X1 and measuring pair 18B is separated from measuring pair 18AA, in the axial direction of lead 10A, by distance X2. In some examples, a distance between respective measuring pairs 18, such as distance X1 and/or X2, may be 4 centimeters (cm). In some examples, a distance between respective measuring pairs 18, such as distance X1 and/or X2, may be between 2 cm and 6 cm. In some examples, a distance between respective measuring pairs 18, such as distance X1 and/or X2, may be other distances not listed above. In some examples, a distance between respective measuring pairs 18 may be substantially the same on lead 10A. For example, distance X1 may equal or substantially equal distance X2. In some examples, a first distance between respective measuring pairs 18 on lead 10A may vary. For example, distance X1 may be different than distance X2. In some examples, a first distance between a measuring pair 18 on lead 10A may be equal to or substantially equal to a second distance between other measuring pairs on lead 10A and may also be different than a third distance between another measuring pairs on lead 10A. For example, distance X1 may be equal to distance X2, but distance X1 may be different than a distance between measuring pair 18AA and measuring pair 18BB.

In some examples, as shown in FIG. 1B, system 100A may include one or more leads 10 (e.g., lead 10B illustrated in FIG. 1B), processing circuitry 110, and/or an external device 108. In some examples, as shown in FIG. 1B, lead 10B may include one or more controllers 30A electrically coupled to one or more electrodes 12, LEDs 14, and/or optical sensors 16 on lead 10B. FIG. 1D shows examples of a controller 30, such as controller 30A or controller 30B. In some examples, controller 30 may include one or more of a power source 32, LED driver circuitry 34 configured to drive LEDs 14, sensing circuitry 35, processing circuitry 36, and/or a wired/wireless communication interface 38. For examples, controller 30A may include a wireless communication interface 38 and controller 30B may include a wired communication interface 38. In some examples, some or all of functionality attributed to processing circuitry 110 described herein may be performed by processing circuitry 38. In some examples, the power source 32 may be primary, rechargeable (e.g., transcutaneously), and/or powered entirely wirelessly/transcutaneously. In some examples, the power source 32 may be include a battery. In some examples, such as shown in FIG. 1B, lead 10B including one or more controllers 30A may be referred to as a wireless lead. In FIG. 1B, a measuring pair may be an electrode 12 and one of an adjacent LED 14 or an adjacent optical sensor 16. In some examples, lead 10B may be in wireless communication with at least one of external device 108, processing circuitry 110, and other devices not pictured in FIG. 1B. In some examples, controller 30A may be separated by distance X3 from an adjacent LED 14, optical sensor 16, or electrode 12 of an adjacent measuring pair 18. In some examples, distance X3, may be between 2 cm and 4 cm. In some examples, distance X3, may be 3 cm. In some examples, distance X3 may be other distances not listed above.

In some examples, as shown in FIG. 1C, a system 100B may include one or more leads 10 (e.g., lead 10C in the illustrated example) and a controller 30B. In some examples, a system 100B may include one or more leads 10 (e.g., lead 10C in the illustrated example) and an external device 108 including controller 30B. In some examples, as shown in FIG. 1C, lead 10C may include one or more contacts 40 configured to electrically couple the lead 10B to a controller 30B. For example, lead 10B, such as one or more electrodes 12, LEDs 14, and/or optical sensors 16 on lead 10C, may be electrically coupled to controller 30B via wires that connect to one or more contacts 40. In some examples, the one or more contacts 40 may extend an axially direction by distance X4. In some examples, distance X4, may be between 8 cm and 12 cm. In some examples, distance X4, may be 10 cm. In some examples, distance X4 may be other distances not listed above. Controller 30B may include a wired communication interface 38. In FIG. 1C, a measuring pair may be an electrode 12 and one of an adjacent LED 14 or an adjacent optical sensor 16. The controller 30B may include processing circuitry 110. In some examples, such as shown in FIG. 1C, lead 10C including one or more contacts 40 to electrically couple to a controller 30B may be referred to as a wired lead.

Processing circuitry 110, in some examples, may include one or more processors that are configured to implement functionality and/or process instructions for execution within a device. For example, processing circuitry 110 may be capable of processing instructions stored in a storage device. Processing circuitry 110 may include, for example, microprocessors, graphical processing units (GPUs), tensor processing units (TPUs), digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 110 may include any suitable structure, whether in hardware, software, firmware, or any combination thereof, to perform the functions ascribed herein to processing circuitry 110. Processing circuitry 110 may represent processing circuitry located within any one or more of external device 108, controller 30B, and/or implantable medical device (IMD) 106. In some examples, processing circuitry 110 includes sensing circuitry configured to generate physiological information from the sensed electrical signals and/or the sensed optical signal(s) of patient 102.

In some examples, lead 10 is configured to sense EEG signals via one or more of the electrodes 12 and configured to sense optical signals, such as NIR signals, via the one or more optical sensors 16. In some examples, processing circuitry 110 may sense, via one or more of the electrodes 12, electrical signal from patient 102, such as during a period of time. Processing circuitry 110 may generate, based on the electrical signals, EEG signal(s). In some examples, processing circuitry 110 may sense, via at least one of optical sensors 16 and using at least one of LEDs 14, optical signals, such as during the period of time processing circuitry senses the electrical signals. For example, processing circuitry 110 may sense, via one or more electrodes 12 and/or one or more optical sensors 16 on lead 10, EEG signal(s) and optical signal(s) simultaneously and/or substantially simultaneously. In some examples, processing circuitry 110 may sense, via one or more electrodes 12 on lead 10, EEG signal(s) during a same period of time that processing circuitry senses, via one or more optical sensors 16 on lead 10, optical signal(s).

In some examples, processing circuitry 110 may determine one or more of blood oxygenation or blood flow, such as cerebral blood flow, of patient 102 based, at least in part, on the optical signal(s) sensed by optical sensors 16. In some examples, processing circuitry 110 may alternatively or additionally determine cerebral metabolic rate of oxygen of patient 102 based, at least in part, on the optical signal(s) sensed by optical sensors 16. In some examples, processing circuitry 110 may determine perfusion pressure, such as ICP, based on one or more of blood flow or blood oxygenation of patient 102 determined via the sensed optical signals. In some examples, processing circuitry 110 may determine perfusion pressure, such as ICP, based on the EEG signal(s) determine via the sensed electrical signals and one or more of blood flow or blood oxygenation of patient 102 determined via the sensed optical signals. Processing circuitry 110 may generate a metric, such as a stoke metric or a pressure metric, indicative of a stroke status or perfusion pressure status of patient 102 based on the generated EEG signal(s) and one or more of the determined cerebral blood flow and/or blood oxygenation. In some examples, processing circuitry 110 may generate a stoke metric indicative of a stroke status of patient 102 based on the generated EEG signal(s) and the perfusion pressure.

While conventional approaches to stroke detection utilizing EEG have relied on data from a large number of EEG electrodes, this disclosure describes that clinically useful stroke determinations can be made utilizing relatively few electrodes, such as via the electrodes 12 carried by one or more leads 10, in combination with relatively few optical sensors, such as via the optical sensors 16 carried by one or more leads 10.

The sensed electrical signals may include features representative of brain function, such as amplitudes of frequencies in one or more frequency bands, such as delta bands, theta bands, alpha bands, beta bands, or gamma bands. Brain signal analysis circuitry, which may be implemented as part of processing circuitry 110 may perform various signal processing to extract these brain features from the sensed electrical signals. In some examples, the electrical signals associated with brain activity may be intermixed with electrical signals associated with cardiac activity (e.g., ECG signals) or signals including components associated with mechanical activity of the heart and skeletal muscle activity (e.g., EMG signals) and artifacts from other electrical sources such as patient movement or external interference. Accordingly, in some embodiments, the sensor data may be filtered or otherwise manipulated to separate the brain activity data (e.g., EEG signals) and ECG signals (or other cardiac signals) from each other and other electrical signals (e.g., EMG signals, etc.). In some examples, IMD or an external device may employ machine learning/adaptive neural network techniques to improve the signal extraction capability (e.g., to filter out or reduce the contribution of ECG signals from the EEG signals). One such methodology is described in “ECG Artifact Removal of EEG signal using Adaptive Neural Network” as published in IEEE Xplore 27 May 2019, which is hereby incorporated by reference in its entirety. Similarly, electrical signals associated with skeletal muscle activity may also be filtered from the EEG sensor data to remove such artifacts.

In some examples, one or more sensors (e.g., electrodes, optical sensors, or any combination thereof) of lead(s) 10 may generate a signal that indicates a parameter of a patient. In some examples, the signal that indicates the parameter includes a plurality of parameter values, where each parameter value of the plurality of parameter values represents a measurement of the parameter at a respective interval of time. The plurality of parameter values may represent a sequence of parameter values, where each parameter value of the sequence of parameter values are collected by processing circuitry 110 at a start of each time interval of a sequence of time intervals. For example, processing circuitry 110 may perform (e.g., control sensing circuitry to sense via lead(s) 10) a parameter measurement in order to determine a parameter value of the sequence of parameter values according to a recurring time interval (e.g., every day, every night, every other day, every twelve hours, every hour, or any other recurring time interval). In this way, processing circuitry 110 may be configured to track a respective patient parameter more effectively as compared with a technique in which a patient parameter is tracked during patient visits to a clinic, since lead(s) 10 is implanted within patient 102 and is configured to perform parameter measurements according to recurring time intervals without missing a time interval or performing a parameter measurement off schedule. Processing circuitry 110 may determine these different parameters separately from the stroke metrics or determine the stroke metrics based at least partially on one or more other parameter measurements.

In some examples, EEG signals fall in the range of approximately 0.5 Hertz (Hz)-approximately 200 Hz. In some examples, EEG signals fall in the range of approximately 0.5 Hertz (Hz)-approximately 500 Hz. Waveforms may be subdivided into bandwidths known as delta (δ), theta (θ), alpha (α), beta (β), and gamma (γ). For example, a delta (δ) band may be between 0.5 Hz and 4 Hz, a theta (θ) band may be between 4 Hz and 8 Hz, an alpha (α) band may be between 8 Hz and 13 Hz, a beta (β) band may be between 13 Hz and 30 Hz, and a gamma (γ) band may be between 30 Hz to 200 Hz. In some examples, γ band may be between 30 Hz to 500 Hz. In some examples, the disclosure describes techniques for detecting stroke that use a ratio of the energies in two of these bands as a metric, e.g., to compare the value of the ratio (or other metric) in a test signal to the ratio in the baseline signal. Example ratios that the techniques of this disclosure may use to detect stroke include a delta-alpha ratio (DAR), delta-theta ratio (DTR), a (delta+theta)/(alpha+beta) ratio (DTABR), a beta-alpha ratio (BAR), a gamma-alpha ratio (GAR), and a burst-suppression ratio (BSR). In some examples, the respective ratios may be signal power ratios between the respective frequency bandwidths. In some examples, a BSR may be a fraction of an EEG signal spent in a suppressed state (e.g., an amplitude of EEG signal being below a suppressed state threshold, such as less than 5 micro volts) over a period of time.

In some examples, features of an EEG signal may include a bandwidth ratio of waveforms of an EEG signal. In some examples, a stroke metric may comprise or be based on a bandwidth ratio of the EEG signal. A bandwidth ratio may be a ratio of bandwidth of the waveforms of the EEG signals, such as delta (δ), theta (θ), alpha (α), beta (β), and gamma (γ). In some examples, bandwidth ratio may include a DTABR, DTR, DAR, or BSR.

In some examples, processing circuitry 110 may generate a stoke metric indicative of a stroke status of patient 102 based on one or more particular bandwidth ratios of the generated EEG signal(s) and one or more of the determined cerebral blood flow and/or blood oxygenation. In some examples, a stroke metric based on an EEG signal and one or more of the determined cerebral blood flow and/or blood oxygenation may include a revised brain symmetry index (rsBSI), derived Brain Symmetry Index (pdBSI), regional attenuation without delta (RAWOD), a spectral score, or other scores.

In some examples, the stroke metric may be indicative of whether or not the patient has experienced a stroke. In some examples, the stroke metric may alternatively or additionally be indicative of whether or not the patient is predicted to experience a stroke in the near future, such as within the next hour, within the next day, or within the next week. The processing circuitry 110 of the system may store the stroke metrics over time. In some examples, the processing circuitry 110 may cause the stroke metrics to be transmitted to an external device periodically or in response to a trigger event, such as detection of a stroke being experienced by the patient. In other examples, processing circuitry 110 may cause the stroke metric to be transmitted to another IMD or external medical device configured to deliver electrical stimulation therapy, mechanical/electrical thrombectomy and/or drug delivery therapy. In other examples, processing circuitry 110 may trigger the generation of stroke metrics in response to a trigger event that indicates the risk for stroke has increased, respectively.

In some examples, the generated stroke metric satisfying a stroke criteria threshold may correspond to a triggering event. In some examples, in response to the generated stroke metric satisfying a stroke criteria threshold, the IMD may cause patient stroke data, such as one or more of the EEG signal(s), the optical signal(s), the cerebral blood flow, and/or the blood oxygenation be sent to another computing device, such as a clinician's computing device, a hospital computing device, a patient's smartphone and/or a server, for further adjudication. In some examples, the further adjudication may include another computing device confirming or denying whether a stroke was detected by the processing circuitry 110 and/or confirming or denying what type of stroke was detected. In some examples, the further adjudication may include another computing device applying an artificial intelligence model (e.g., machine learning, neural networks, etc.) to patient stroke data to confirm or deny whether a stroke was detected by the processing circuitry and/or confirm or deny what type of stroke was detected.

In some examples, one or more leads 10 being able to sense EEG and sense optical signals that are substantially simultaneous to the sensed EEG signals may enable processing circuitry 110 to determine blood flow and/or blood oxygenation parameter(s) useful for detection or prediction of stroke and/or discrimination of ischemic and hemorrhagic stroke in high-risk patients. In addition, one or more leads 10 being able to sense EEG and sense optical signals that are substantially simultaneous to the sensed EEG signals may enable processing circuitry 110 to determine blood flow and/or blood oxygenation to detect stroke and/or discriminate ischemic and hemorrhagic stroke in high-risk patients. Subcutaneous or subgaleal implantation of lead(s) 10 as described herein may be less invasive and associated with less risk of infection than some conventional techniques, such as with an invasive drain.

The electrodes 12 may be configured to detect signals that enable processing circuitry, such as processing circuitry 110, of a computing device communicatively coupled to lead 10 to determine current values of metrics, such as stroke metrics, associated with the brain and/or cardiovascular functions of a patient. In some examples, the electrodes 12 are configured to detect a signal indicative of an electric potential of the tissue surrounding the lead 10.

Each of the stroke metrics may be indicative of the likelihood (or risk) that patient 102 has experienced, or is experiencing, a stroke, respectively. For example, each stroke metric may include a numerical value representative of the probability that patient 102 has experienced a stroke. Processing circuitry 110 may then compare the metric to a respective threshold or monitor a relative change in the metric value over time to determine whether or not a stroke occurred. In other examples, the stroke may be a binary value that indicates no event occurred or that an event did occur. In some examples, IMD 106 may generate each stroke metric based on sensed data other than the sensed electrical signals from the carried electrodes on the housing of IMD 106.

Processing circuitry 110 may generate the metrics, such as stroke metrics, at the same or different frequencies. For example, for a patient who is considered a high-risk patient, processing circuitry 110 may generate metrics, such as stroke metrics, every 30 minutes, hourly or daily. These time periods are examples, and the generation of metrics, such as stroke metrics, are not limited to the periods discussed above. In some examples, these frequencies may refer to the frequency at which the sensing circuitry generates appropriate information from which the metric is determined. In other examples, processing circuitry 110 may continually generate physiological information from which metrics, such as stroke metrics, can be determined. However, the frequency may refer to how often processing circuitry 110 generates the metric, such as the stroke metric, from the physiological information. Continually generating physiological information may include sensing physiological signal and other generation of physiological information on a periodic and/or triggered basis without user intervention.

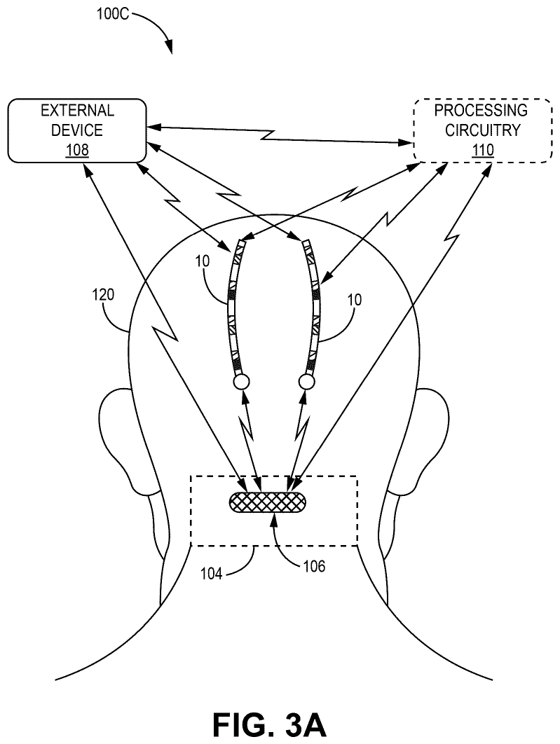

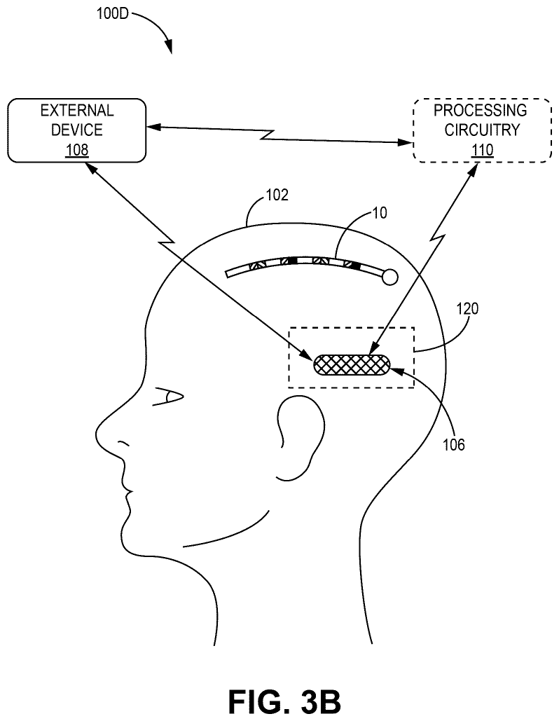

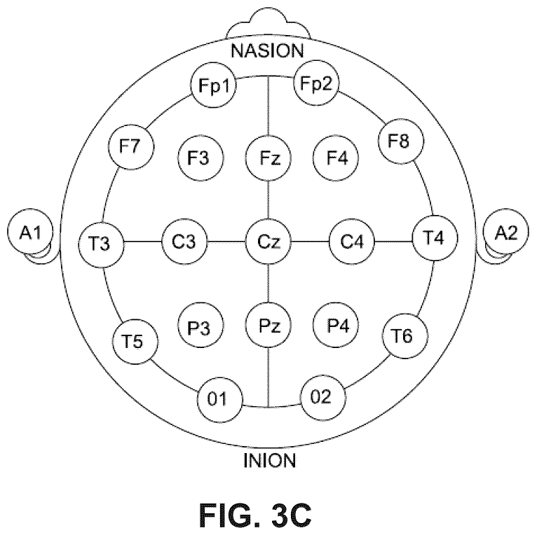

FIGS. 2A-2D show example target areas that leads 10D-10H may be positioned on a head of patient 102. In some examples, leads 10D-10H may be similar or the same as one or more of leads 10A-10C. In some examples, leads 10D-10H may be similar or the same as each other. As shown in FIG. 2A, lead 10D may be positioned on a left side (e.g., side of the left-arm and/or left leg) of the top of the head of patient 102 while lead 10E may be positioned on a right side (e.g., the side of the right arm and/or right leg) of the top of the head of patient 102. As shown in FIG. 2A, the respective leads 10D, 10E are positioned to have one or more measuring pairs (e.g., 12A/14A, 12B/16A, 12C/14B, and 12D/16B, referred to as measuring pairs 18A, 18B, 18AA, 18BB) positioned in each hemisphere, such as left hemisphere LH and right hemisphere RH of a head of patient 102. In addition, as shown in FIG. 2A, one or more measuring pairs may be positioned in the front hemisphere FH and the back hemisphere BH of the head of patient 102. In some examples, in the configuration shown in FIG. 2A, the electrodes of lead 10D may detect electrical activity that corresponds to brain activity in the P3, C3, and/or F3 regions (as shown in FIG. 3C) and/or the electrodes of lead 10E may detect electrical activity that corresponds to brain activity in the P4, C4, and/or F4 regions (as shown in FIG. 3C).

In some examples, as shown in FIGS. 2B-2C, leads 10F and 10G may be positioned on a side of a head of patient 102, such as a right side, as shown in FIG. 2B, and on a left side, as shown in FIG. 2C. Specifically, in this particular configuration, the electrodes of lead 10F may detect electrical activity that corresponds to brain activity in the T4, T6, and/or F8 regions (as shown in FIG. 3C) and/or the electrodes of lead 10G may detect electrical activity that corresponds to brain activity in the T3, T5, and/or F7 regions (as shown in FIG. 3C). In some examples, as shown in FIG. 2D, lead 10H may be positioned on a top of a head of patient 102. While the particular electrodes on leads 10F, 10G, and 10H are not identified in FIGS. 2B-2D, leads 10F-10H may be arranged similarly to leads 10A, 10D, and/or 10E, as shown in FIG. 1A and FIG. 2A.

In some examples, lead 10 may be configured to sense Auditory Evoked Potential (AEP) and/or Visual Evoked Potential (VEP). For example, patient 102 may be visually stimulated, such as with goggle or an eye piece, and lead 10 may be configured to sense VEP in response to visual stimuli being applied to patient 102. In some examples, patient 102 may be auditorily stimulated, such as via a device with a speaker (e.g., an earpiece or earbud), and lead 10 may be configured to sense AEP in response to auditory stimuli being applied to patient 102. In some examples, patient 102 may be both visually and auditorily stimulated and lead 10 may be configured to sense VEP and AEP in response to visual and auditory stimuli being applied to patient 102.

In some examples, when processing circuitry 110 determines a generated stroke metric based on EEG signal(s) and one or more of the cerebral blood flow or blood oxygenation indicates patient 102 had a stroke, processing circuitry 110 may determine or confirm patient 102 had a stroke based on sensed VEP and/or AEP in response to visual and auditory stimuli being applied to patient 102. In some examples, if the sensed VEP and/or the sensed AEP satisfy a respective evoked potential threshold, processing circuitry 110 determines patient 102 suffered stroke. For example, in response to one or more of visual stimuli or audio stimuli being applied to the patient, such as via googles and/or earbuds, processing circuitry 110 may be configured to sense, via at least one of the plurality of electrodes 12 and/or one of the optical sensors 16, VEP and/or auditory evoked potential AEP. Processing circuitry 110 may be configured to determine whether one or more of the VEP or the AEP satisfies a stroke threshold. In response to processing circuitry 110 determining one or more of the VEP or the AEP satisfies a stroke threshold, processing circuitry 110 may be configured to determine and/or confirm a stroke occurred in the patient.

In some examples, in response to processing circuitry 110 determining patient 102 suffered stroke based on the sensed VEP and/or the sensed AEP satisfying a respective evoked potential threshold, processing circuitry 110 may perform polyspectral analysis (PSA) on a particular EEG signal sensed by one or more leads 10. For example, the particular EEG signal may include a duration for a period of time beginning before visual and/or audio stimuli are applied to patient 102, such as, but not limited to, 1 to 10 seconds before the stimuli are applied, and ending at a timepoint after the visual and/or audio stimuli are applied to the patient 102, such as, but not limited to, 1 to 30 seconds after stimuli are applied. In some examples, processing circuitry 110 may determine whether a potential stroke, such as a potential stroke that was identified by the generated stroke metric, is a stroke by determining whether the PSA of the particular EEG signal satisfies a stroke threshold.

FIG. 3A is a conceptual diagram of a system 100C configured to detect stroke in accordance with some examples of the present disclosure. In some examples, system 100C may further include an IMD 106 to be used in conjunction with one or more leads 10 to detect physiological parameters. For example, system 100C may be used with one or more leads 10, as discussed above with reference to FIGS. 1A-2D, an implantable medical device (IMD) 106, which may be in wireless communication with at least one of external device 108, processing circuitry 110, one or more leads 10, and other devices not pictured in FIG. 3A. For example, a computing device (not illustrated in FIG. 3A) may include at least a portion of processing circuitry 110, the computing device configured for communication with IMD 106, and external device 108. As shown in FIG. 3A, IMD 106 may be located in target region 104. Target region 104 can be a rear portion of a user's neck or at the base of the skull. Although IMD 106 may be implanted at a location generally centered with respect to the head, neck, or target region 104, IMD 106 may be implanted in an off-center location in order to obtain desired vectors from the electrodes carried on the housing of IMD 106. In other examples, target region may be located at other positions of patient, such as near the user's temple(s) (e.g., above the ear(s)) and/or over the temporal portion of the skull. IMD 106 can be disposed in target region 104 either via implantation (e.g., subcutaneously) or by being placed over the patient's skin with one or more electrodes of IMD 106 being in direct contact with the patient's skin at or adjacent the target region 104. In some examples the system may include plurality of IMDs 106, such as two or more IMDs 106 configured to individually and/or cooperatively detect stroke in accordance with examples of the present disclosure.

IMD 106 may extract features from EEG signals indicative of brain activity or cardiac activity. IMD 106 may then determine whether or not the patient has experienced a stroke based on these extracted features. In some examples, IMD 106 takes the form of a LINQ™ Insertable Cardiac Monitor (ICM), available from Medtronic, Inc., of Minneapolis, Minnesota. The example techniques may additionally, or alternatively, be used with a medical device not illustrated in FIG. 3A such as another type of IMD, a patch monitor device, a wearable device (e.g., smart watch), or another type of external medical device.

Clinicians sometimes diagnose a patient (e.g., patient 102) with medical conditions and/or determine whether a condition of patient 102 is improving or worsening based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patient is in a clinic for a medical appointment. However, in some examples, events that may change a condition of a patient, such as administration of a therapy, may occur outside of the clinic. As such, in these examples, a clinician may be unable to observe the physiological markers needed to determine whether an event, such as a stroke, has changed a medical condition of the patient and/or determine whether a medical condition of the patient is improving or worsening while monitoring one or more physiological signals of the patient during a medical appointment. In the example illustrated in FIG. 3A, IMD 106 is implanted within patient 102 to continuously record one or more physiological signals of patient 102 over an extended period of time.

In some examples, IMD 106 includes a plurality of electrodes. The plurality of electrodes is configured to detect signals that enable processing circuitry of IMD 106 to determine current values of metrics associated with the brain and/or cardiovascular functions of patient 102. In some examples, the plurality of electrodes of IMD 106 are configured to detect a signal indicative of an electric potential of the tissue surrounding the IMD 106. Moreover, IMD 106 may additionally or alternatively include one or more optical sensors, accelerometers, impedance sensors, respiration sensors, temperature sensors, chemical sensors, light sensors, pressure sensors, and acoustic sensors, in some examples. Such sensors may detect one or more physiological parameters indicative of a patient condition.

External device 108 may be a hand-held computing device with a display viewable by the user and an interface for providing input to external device 108 (e.g., a user input mechanism). In some examples, external device 108 may be a smartphone, smart watch, smart glasses, or other personal smart device. In some examples, external device 108 may be a smart device of patient 102. For example, external device 108 may include a small display screen (e.g., a liquid crystal display (LCD) or a light emitting diode (LED) display) that presents information to the user. In addition, external device 108 may include a touch screen display, keypad, buttons, a peripheral pointing device, voice activation, or another input mechanism that allows the user to navigate through the user interface of external device 108 and provide input. If external device 108 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device 108 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device. In some examples, external device 108 may be controller 30.

When external device 108 is configured for use by the clinician, external device 108 may be used to transmit instructions to IMD 106. Example instructions may include requests to set electrode combinations for sensing and any other information that may be useful for programming into lead(s) 10 and/or IMD 106. To program controller 30 and/or IMD 106, the clinician may configure and store operational parameters of controller 30 coupled to lead(s) 10 and/or IMD 106 within IMD 106 with the aid of external device 108. In some examples, external device 108 assists the clinician in the configuration of lead(s) 10 and/or IMD 106 by providing a system for identifying potentially beneficial operational parameter values.

Whether external device 108 is configured for clinician or patient use, external device 108 is configured to communicate with lead(s) 10 and/or IMD 106 and, optionally, another computing device (not illustrated by FIG. 3A), via wireless communication. External device 108, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies). In some examples, external device 108 is a smartphone of patient 102 and/or a watch or other wearable computing device, which may communicate with lead(s) 10 and/or IMD 106, e.g., via Bluetooth™. In some examples, external device 108 is configured to communicate with a computer network, such as the Medtronic CareLink® Network developed by Medtronic, plc, of Dublin, Ireland. For example, external device 108 may send data, such as data received from lead(s) 10 and/or IMD 106, to another external device such as a smartphone, a tablet, or a desktop computer, and the other external device may in turn send the data to the computer network. In other examples, external device 108 may directly communicate with the computer network without an intermediary device.

Processing circuitry 110, in some examples, may include one or more processors that are configured to implement functionality and/or process instructions for execution within lead(s) 10 and/or IMD 106. For example, processing circuitry 110 may be capable of processing instructions stored in a storage device. Processing circuitry 110 may include, for example, microprocessors, graphical processing units (GPUs), tensor processing units (TPUs), digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 110 may include any suitable structure, whether in hardware, software, firmware, or any combination thereof, to perform the functions ascribed herein to processing circuitry 110.

Processing circuitry 110 may represent processing circuitry located within any one or more of IMD 106, external device 108, and/or controller 30. In some examples, processing circuitry 110 may be entirely located within a housing of IMD 106. In other examples, processing circuitry 110 may be entirely located within a housing of external device 108. In other examples, processing circuitry 110 may be located within any one or combination of IMD 106, external device 108, controller 30, and another device or group of devices that are not illustrated in FIG. 3A. As such, techniques and capabilities attributed herein to processing circuitry 110 may be attributed to any combination of IMD 106, external device 108, and other devices that are not illustrated in FIG. 3A.

Medical device system 100C of FIG. 3A is an example of a system configured to collect electrical signals and generate stroke metrics according to one or more techniques of this disclosure. In some examples, processing circuitry 110 includes sensing circuitry configured to generate physiological information from the sensed electrical signal of patient 102. In one example, an electrical signal is sensed via one or more electrode combinations of IMD 106 and/or one or more electrode 12 combinations of lead(s) 10, and/or one or more optical sensor 16 combinations of lead(s) 10. An electrical signal is representative of electrical activity of the brain, heart, or other physiological functions as measured by electrodes implanted within the body.

In some examples, IMD 106 includes one or more accelerometers. An accelerometer of IMD 106 may collect an accelerometer signal which reflects a measurement of any one or more of a motion of patient 102, a posture of patient 102 and a body angle of patient 102. In some cases, the accelerometer may collect a three-axis accelerometer signal indicative of patient 102's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 102 along a vertical axis, the lateral axis accelerometer signal vector may represent an acceleration of patient 102 along a lateral axis, and the frontal axis accelerometer signal vector may represent an acceleration of patient 102 along a frontal axis. In some cases, the vertical axis substantially extends along a torso of patient 102 when patient 102 from a neck of patient 102 to a waist of patient 102, the lateral axis extends across a chest of patient 102 perpendicular to the vertical axis, and the frontal axis extends outward from and through the chest of patient 102, the frontal axis being perpendicular to the vertical axis and the lateral axis.

IMD 106 may measure a set of parameters including an impedance (e.g., subcutaneous impedance measured via electrodes, an intrathoracic impedance or an intracardiac impedance) of patient 102, a respiratory rate of patient 102 during night hours, a respiratory rate of patient 102 during day hours, a heart rate of patient 102 during night hours, a heart rate of patient 102 during day hours, an atrial fibrillation (AF) burden of patient 102, a ventricular rate of patient 102 while patient 102 is experiencing AF, or any combination thereof. Processing circuitry 110 may analyze any one or more of the set of parameters in order to determine whether or not the patient is experiencing stroke, and may indicate an efficacy of a treatment program administered to patient 102. In some examples, pulsatile signals sensed optically or mechanically, e.g., via the electrodes, an optical sensor, accelerometer, pressure sensor, impedance sensor, or heart sound sensor, from the scalp vasculature may provide a surrogate for an ECG or other cardiac electrical activity signal. In some examples, the treatment program may include treatment delivered by one or more medical devices such as ICDs with intravascular or extravascular leads, pacemakers, CRT-Ds, neuromodulation devices, LVADs, implantable sensors, orthopedic devices, or drug pumps. Additionally, or alternatively, the treatment program may include in-clinic treatments administered by medical professionals, prescribed pharmaceutical regimens, treatments administered by one or more external medical devices, or any combination thereof. In any case, processing circuitry 110 may determine the efficacy of the treatment program by determining a time in which the treatment program is administered (e.g., including a time in which the treatment program begins and/or a time in which the treatment program ends) and analyzing values of any one or combination of the set of parameters relative to the time in which the treatment program is administered. Alternatively, in some examples, processing circuitry 110 may determine the efficacy of a treatment program by evaluating one or more parameters on a rolling basis in order to determine whether the one or more parameters have changed over a period of time.

In some examples, one or more sensors (e.g., electrodes, motion sensors, optical sensors, temperature sensors, or any combination thereof) of IMD 106 may generate a signal that indicates a parameter of a patient. In some examples, the signal that indicates the parameter includes a plurality of parameter values, where each parameter value of the plurality of parameter values represents a measurement of the parameter at a respective interval of time. The plurality of parameter values may represent a sequence of parameter values, where each parameter value of the sequence of parameter values are collected by IMD 106 at a start of each time interval of a sequence of time intervals. For example, IMD 106 may perform a parameter measurement in order to determine a parameter value of the sequence of parameter values according to a recurring time interval (e.g., every day, every night, every other day, every twelve hours, every hour, or any other recurring time interval). In this way, IMD 106 may be configured to track a respective patient parameter more effectively as compared with a technique in which a patient parameter is tracked during patient visits to a clinic, since IMD 106 is implanted within patient 102 and is configured to perform parameter measurements according to recurring time intervals without missing a time interval or performing a parameter measurement off schedule.

IMD 106 may be referred to as a system or device. In one example, IMD 106 may include a memory, a plurality of electrodes carried by the housing of IMD 106, sensing circuitry configured to sense, via at least two electrodes of the plurality of electrodes, electrical signals from patient 10 and generate, based on the electrical signals, physiological information. The housing of IMD 106 carries the plurality of electrodes and contains, or houses, both of the sensing circuitry and the processing circuitry. In this manner, IMD 106 may be referred to as a leadless sensing device because the electrodes are carried directly by the housing instead of by any leads that extend from the housing. In some examples, however, sensor device 106 may include one or more sensing leads extending therefrom and into the tissue of the patient; such lead(s) may be employed instead of or in addition to the electrodes of sensor device 106, and may perform any of the functions attributed herein to the electrodes.

The physiological data can include electrical brain activity data and/or electrical heart activity data. In some examples, the plurality of electrodes are configured to detect brain activity data corresponding to activity in at least one of a P3, Pz, or P4 brain region, which is at the back of the head or upper neck region as shown in FIG. 3C. In this manner, the housing of IMD 106 may be configured to be disposed at or adjacent to a rear portion of a neck or skull of patient 102. The housing of IMD 106 may be configured to be implanted within patient 104, such as implanted subcutaneously. In other examples, the housing of IMD 106 may be configured to be disposed on an external surface of skin of patient 102.

In some examples, IMD 106 may include a single sensing circuitry configured to generate, from the sensed electrical signals, information that includes both the electrical brain activity data (e.g., EEG data) and the electrical heart activity data (e.g., ECG data) or cardiac contraction data. In other examples, the processing circuity of IMD 106 may include separate hardware that generates different information from the sensed electrical signals. For example, IMD 106 may include first circuitry configured to generate the electrical brain activity from the electrical signals and second circuitry different from the first circuitry and configured to generate the electrical heart activity data from the electrical signals. Even with the first and second circuitry configured to generate different information, or data, in some examples, sensed electrical signals may be conditioned or processed by one or more electrical components (e.g., filters or amplifiers) prior to being processed by the first and second circuitry. In some examples, electrical brain activity data may include features, such as spectral features, indicative of the strength of signals in various frequency bands or at various frequencies. In some examples, electrical heart activity data may include features such as the timing and/or amplitude of P-waves, R-waves, or any other features representative of heart function.

In one example, IMD 106 may include one or more accelerometers within the housing. The accelerometer may be configured to generate accelerometer signal, which may be stored processed as motion/posture data, representative of posture and/or motion of patient 102. IMD 106 may then be configured to determine one or more of a posture of a patient or activity level of a patient based on the generated accelerometer signal.

In some examples, the physiological information generated from the sensed electrical signals may include ECG information. IMD 106 may extract various features from the ECG information, such as heart rate, heart rate variability, etc.

FIG. 3B is a conceptual diagram of a system 100D configured to detect stroke in accordance with examples of the present disclosure. System 100D may be substantially similar to system 100C of FIG. 3A. However, system 100D may be configured to have IMD 106 be implanted in target region 120 which is located on the side of the head posterior of the temple of patient 102, e.g., above the ear and/or over the temporal portion of the cranium. IMD 106 implanted at target region 120 may be configured to generate physiological metrics based on electrical signals sensed in this area. In such examples, the electrodes of IMD 106 may detect electrical activity that corresponds to brain activity in the T3 region (as shown in FIG. 3C), or T4 region if implanted on the other side of the patient's head, or both of two or more sensor devices are implanted bilaterally at temporal regions. In some examples, IMD 106 may need to employ different filters or other processing or signal conditioning techniques than those at target region 104 due to different types of noise at target region 120, such as muscle activity due to mandible movement or other types of electrical activity.

Systems (e.g., processing circuitry) described herein may use different localized EEG signals to localize the stroke or other brain event, e.g., to a particular hemisphere, or for other purposes related to diagnosing such events as described herein. In some examples, e.g., as described with respect to FIG. 2G, a single IMD may include electrodes coupled thereto via extensions, which may be positioned in different hemispheres or other regions to similar acquire localized EEG signals.

FIG. 3C is a diagram of the 10-20 map for EEG sensor measurements. As shown in FIG. 3C, various locations on the head of patient 12 may be targeted using the electrodes carried by IMD 106. In some examples, the various locations may include one or more of A1, A2, Fp1, Fp2, F7, F3, Fz, F4, F8, T3, C3, Cz, C4, T4, T5, P3, Pz, P6, T6, 01, and/or 02. At the back of the head, such as in target region 104 of FIG. 3A, IMD 106 may sense electrical signals at least one of P3, Pz or P4. At the side of the head, such as in target region 120 of FIG. 3B, IMD 106 may sense electrical signals at least one of F7, T3, or T5 adjacent the left hemisphere of the brain. In addition or alternatively, IMD 106 may sense electrical signals at least one of F8, T4, or T6 adjacent the right hemisphere of the brain.

In some examples, IMD 106 may be one or more of the sensor devices described in accordance with U.S. application Ser. No. 17/176,504 by Schulhauser et al., entitled “SYSTEM AND METHOD FOR DETECTING STROKES,” which is incorporated herein by reference in its entirety.

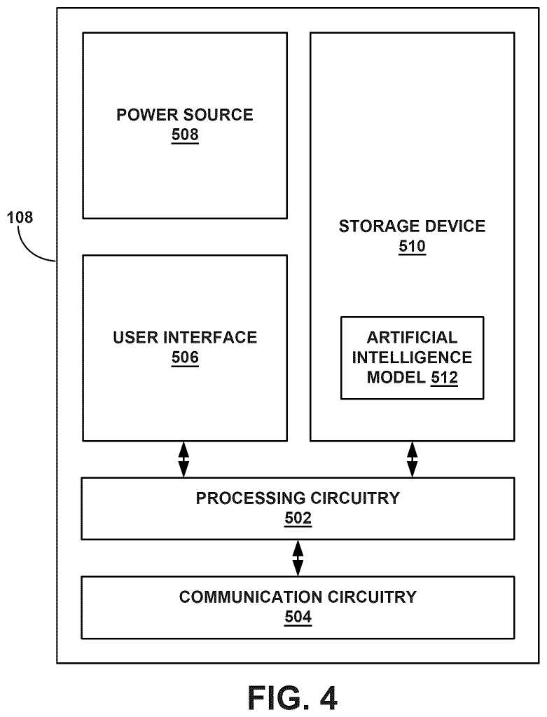

FIG. 4 is a block diagram of an example external device 108 configured to communicate with any of the lead(s) 10/controller 30, IMD 106, or sensor device described herein. In the example of FIG. 4, external device 108 includes processing circuitry 502, communication circuitry 504, storage device 510, user interface 506, and power source 508.

Processing circuitry 502, in one example, may include one or more processors that are configured to implement functionality and/or process instructions for execution within external device 108. For example, processing circuitry 502 may be capable of processing instructions stored in storage device 510. Processing circuitry 502 may include, for example, microprocessors, GPUs, TPUs, DSPs, ASICs, FPGAs, or equivalent discrete or integrated logic circuitry, or a combination of any of the foregoing devices or circuitry. Accordingly, processing circuitry 502 may include any suitable structure, whether in hardware, software, firmware, or any combination thereof, to perform the functions ascribed herein to processing circuitry 502. In some examples, some or all of functionality attributed to processing circuitry 110 described herein may be performed by processing circuitry 502.

Communication circuitry 504 may include any suitable hardware, firmware, software or any combination thereof for communicating with another device, such as IMD 106 and/or lead(s) 10/controller 30. Under the control of processing circuitry 502, communication circuitry 504 may receive downlink telemetry from, as well as send uplink telemetry to, IMD 106 and/or lead(s) 10/controller 30, or another device. In other examples, communication circuitry 504 may also employ TCC for communicating with other devices.

Storage device 510 may be configured to store information within external device 108 during operation. Storage device 510 may include a computer-readable storage medium or computer-readable storage device. In some examples, storage device 510 includes one or more of a short-term memory or a long-term memory. Storage device 510 may include, for example, RAM, dynamic random access memories (DRAM), static random access memories (SRAM), magnetic discs, optical discs, flash memories, or forms of electrically programmable memories (EPROM) or EEPROM. In some examples, storage device 510 is used to store data indicative of instructions for execution by processing circuitry 502. Storage device 510 may be used by software or applications running on external device 108 to temporarily store information during program execution. In some examples, storage device 510 may include an artificial intelligence model 512 (e.g., machine learning, neural networks, etc.) stored on the storage device 510.

Data exchanged between external device 108 and IMD 106 and/or lead(s) 10/controller 30 may include operational parameters. External device 108 may transmit data including computer readable instructions which, when implemented by IMD 106 and/or lead(s) 10/controller 30, may control IMD 106 and/or lead(s) 10/controller 30 to change one or more operational parameters and/or export collected data. For example, processing circuitry 502 may transmit an instruction to IMD 106 and/or lead(s) 10/controller 30 which requests IMD 106 and/or lead(s) 10/controller 30 to export collected data (e.g., data corresponding to one or more of the physiological information, stroke metrics, EEG signal(s) and/or optical signal(s)) to external device 108. In turn, external device 108 may receive the collected data from IMD 106 and/or lead(s) 10/controller 30 and store the collected data in storage device 510. Additionally, or alternatively, processing circuitry 502 may export instructions to IMD 106 and/or lead(s) 10/controller 30 requesting IMD 106 and/or lead(s) 10/controller 30 to update electrode and/or optical sensor combinations for stimulation or sensing.

A user, such as a clinician or patient 102, may interact with external device 108 through user interface 506. User interface 506 includes a display (not shown), such as an LCD or LED display or other type of screen, with which processing circuitry 502 may present information related to lead(s) 10 and/or IMD 106 (e.g., stroke metrics). In addition, user interface 506 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 502 of external device 108 and provide input. In other examples, user interface 506 also includes audio circuitry for providing audible notifications, instructions or other sounds to patient 102, receiving voice commands from patient 102, or both. Storage device 510 may include instructions for operating user interface 506 and for managing power source 508.

Power source 508 is configured to deliver operating power to the components of external device 108. Power source 508 may include a battery and a power generation circuit to produce the operating power. In some examples, the battery is rechargeable to allow extended operation. Recharging may be accomplished by electrically coupling power source 508 to a cradle or plug that is connected to an alternating current (AC) outlet. In addition, recharging may be accomplished through proximal inductive interaction between an external charger and an inductive charging coil within external device 108. In other examples, traditional batteries (e.g., nickel cadmium or lithium ion batteries) may be used. In addition, external device 108 may be directly coupled to an alternating current outlet to operate.

In some examples, external device 108 may provide an alert to the patient or another entity (e.g., a call center) based on a stroke indication. In some examples, user interface 506 may provide an interface for presenting an alert of the detection, prediction, or classification of the condition, e.g., stroke, and for a user, e.g., the patient, a caregiver, or a clinician, to provide input overriding the detection, prediction, or classification. In this manner, systems as described herein may avoid unnecessary emergency activity resulting from a false detection by the system.

In some examples, external device 108 may receive patient data, such as one or more of EEG signal(s) and/or optical signal(s), such as from lead(s) 10/controller 30, to further adjudicate a generated stroke metric satisfying a stroke criteria threshold in IMD 106. In some examples, external device 108 may receive patient data, such as one or more of EEG signal(s) and/or accelerometer signal(s), such as from IMD 106, to further adjudicate a generated stroke metric satisfying a stroke criteria threshold based on one or more of EEG signal(s) and/or optical signal(s) received from lead(s) 10/controller 30. In some examples, external device 108 may apply an artificial intelligence model 512 (e.g., machine learning model, neural networks, etc.) stored in storage device 510 to the received patient data to confirm or deny whether a stroke was detected by lead(s) 10/controller 30 and/or IMD 106 and/or confirm or deny what type of stroke was detected by lead(s) 10/controller 30 and/or IMD 106.

Processing circuitry 502 may be configured to execute an artificial intelligence (AI) engine that operates according to one or more models, such as artificial intelligence models 512. AI models 512 may include any number of different types of AI models, such as machine learning models. Some examples of machine learning models include neural networks, deep neural networks, convolution neural networks, recurrent neural networks, such as long short-term memory networks, dense neural networks, and the like. In some examples, various feature inputs to the AI engine may be fed as direct inputs to different layers in a network and not necessarily prior to the convolution layers. The techniques described in this disclosure are also applicable to other types of AI models, including rule-based models, finite state machines, and the like.

Machine learning may generally enable a computing device to analyze input data and identify an action to be performed responsive to the input data. Each artificial intelligence model may be trained using training data that reflects likely input data. The training data may be labeled or unlabeled (meaning that the correct action to be taken based on a sample of training data is explicitly stated or not explicitly stated, respectively).

The training of the artificial intelligence model may be guided (in that a designer, such as a computer programmer, may direct the training to guide the artificial intelligence model to identify the correct action in view of the input data) or unguided (in that the artificial intelligence model is not guided by a designer to identify the correct action in view of the input data). In some instances, the artificial intelligence model is trained through a combination of labeled and unlabeled training data, a combination of guided and unguided training, or possibly combinations thereof. Examples of machine learning include nearest neighbor, naïve Bayes, decision trees, linear regression, support vector machines, neural networks, k-Means clustering, Q-learning, temporal difference, deep adversarial networks, evolutionary algorithms or other supervised, unsupervised, semi-supervised, or reinforcement learning algorithms to train one or more models.

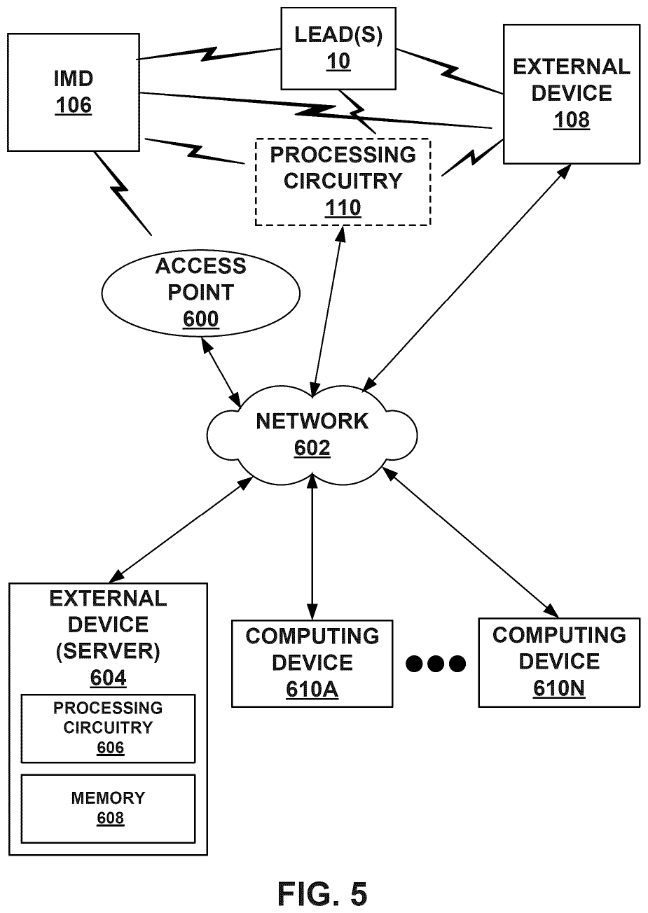

FIG. 5 is a block diagram illustrating an example system that includes an access point 600, a network 602, external computing devices, such as a server 604, and one or more other computing devices 610A-610N, which may be coupled to lead(s) 10/controller 30, IMD 106, external device 108, and processing circuitry 110 via network 602, in accordance with one or more techniques described herein. In this example, IMD 106 may use communication circuitry to communicate with external device 108 via a first wireless connection, and to communicate with an access point 600 via a second wireless connection. In the example of FIG. 5, access point 600, external device 108, server 604, and computing devices 610A-610N are interconnected and may communicate with each other through network 602.

Access point 600 may include a device that connects to network 602 via any of a variety of wired or wireless network connections, such as telephone dial-up, digital subscriber line (DSL), or cable modem connections. In other examples, access point 600 may be coupled to network 602 through different forms of connections, including wired or wireless connections. In some examples, access point 600 may be a user device, such as a tablet or smartphone, that may be co-located with the patient. As discussed above, IMD 106 may be configured to transmit data, such as any one or combination of an EEG signal, an accelerometer signal, and a tissue impedance signal to external device 108. As discussed above, lead(s) 10/controller 30 may be configured to transmit data, such as any one or combination of an EEG signal and an optical signal to external device 108. In addition, access point 600 may interrogate IMD 106, such as periodically or in response to a command from the patient or network 602, in order to retrieve parameter values determined by processing circuitry of IMD 106, or other operational or patient data from IMD 106. Access point 600 may then communicate the retrieved data to server 604 via network 602.

In some cases, server 604 may be configured to provide a secure storage site for data that has been collected from lead(s) 10/controller 30, IMD 106, external device 108, and/or processing circuitry 110. In some cases, server 604 may assemble data in web pages or other documents for viewing by trained professionals, such as clinicians, via computing devices 610A-610N. One or more aspects of the illustrated system of FIG. 5 may be implemented with general network technology and functionality, which may be similar to that provided by the Medtronic CareLink® Network developed by Medtronic, Inc., of Minneapolis, Minnesota.

Server 604 may include processing circuitry 606. Processing circuitry 606 may include fixed function circuitry and/or programmable processing circuitry. In some examples, some or all of functionality attributed to processing circuitry 110 described herein may be performed by processing circuitry 606. Processing circuitry 606 may include any one or more of a microprocessor, a GPU, a TPU, a controller, a DSP, an ASIC, an FPGA, or equivalent discrete or analog logic circuitry. In some examples, processing circuitry 606 may include multiple components, such as any combination of one or more microprocessors, one or more controllers, one or more DSPs, one or more ASICs, or one or more FPGAs, as well as other discrete or integrated logic circuitry. The functions attributed to processing circuitry 606 herein may be embodied as software, firmware, hardware or any combination thereof. In some examples, processing circuitry 606 may perform one or more techniques described herein based on an EEG signal, an optical signal, EGM signal, impedance signal, an accelerometer signal, or other sensor signals received from IMD 106, or parameter values determined based on such signals sensed by lead(s) 10 and/or IMD 106 and received from lead(s) 10//controller 30 and/or IMD 106, as examples. For example, processing circuitry 606 may perform one or more of the techniques described herein to generate a personalized baseline stroke score, determine a second stroke score, and generate a stroke metric indicative of a stroke status of the patient based on a comparison of the personalized baseline stroke score to the second stroke score.

Server 604 may include memory 608. Memory 608 includes computer-readable instructions that, when executed by processing circuitry 606, cause IMD 106 and processing circuitry 606 to perform various functions attributed to IMD 106 and processing circuitry 606 herein. Memory 608 may include any volatile, non-volatile, magnetic, optical, or electrical media, such as RAM, ROM, NVRAM, EEPROM, flash memory, or any other digital media.

In some examples, one or more of computing devices 610A-610N (e.g., device 610A) may be a tablet or other smart device located with a clinician, by which the clinician may program, receive alerts from, and/or interrogate IMD 106. For example, the clinician may access data corresponding to any one or combination of sensed physiological signals, an EEG signal, an optical signal, an accelerometer signal, stroke metrics, and other types of signals collected by lead(s) 10, IMD 106, or parameter values determined by processing circuitry 110, external device 108 and/or IMD 106 based on such signals, through device 610A, such as when patient 102 is in between clinician visits, to check on a status of a medical condition. In some examples, the clinician may enter instructions for a medical intervention for patient 102 into an app in device 610A, such as based on a status of a patient condition determined by IMD 106, external device 108, processing circuitry 110, or any combination thereof, or based on other patient data known to the clinician. Device 610A then may transmit the instructions for medical intervention to another of computing devices 610A-610N (e.g., computing device 610N) located with patient 102 or a caregiver of patient 102. For example, such instructions for medical intervention may include an instruction to change a drug dosage, timing, or selection, to schedule a visit with the clinician, or to seek medical attention. In further examples, device 610B may generate an alert to patient 102 based on a status of a medical condition of patient 102 determined by processing circuitry 110, external device 108, and/or IMD 106, which may enable patient 102 proactively to seek medical attention prior to receiving instructions for a medical intervention. In this manner, patient 102 may be empowered to take action, as needed, to address his or her medical status, which may help improve clinical outcomes for patient 102.

In further examples, device 610B may be configured to transmit alert messages to computing devices 610C associated with one or more care providers via network 602. Care providers may include emergency medical systems (EMS) and hospitals, and may include particular departments within a hospital, such as an emergency department, catheterization lab, or a stroke response department. In further examples, device 610B may provide patient-specific care recommendations (e.g., potential drug therapy for prevention or therapy of stroke and/or electrical/mechanical thrombectomy). The ability of the system to detect the stroke with adequate sensitivity and specificity may, for example, guide EMS care giver to what they can expect when they arrive on the scene and how best to treat the presenting or soon to present stroke.

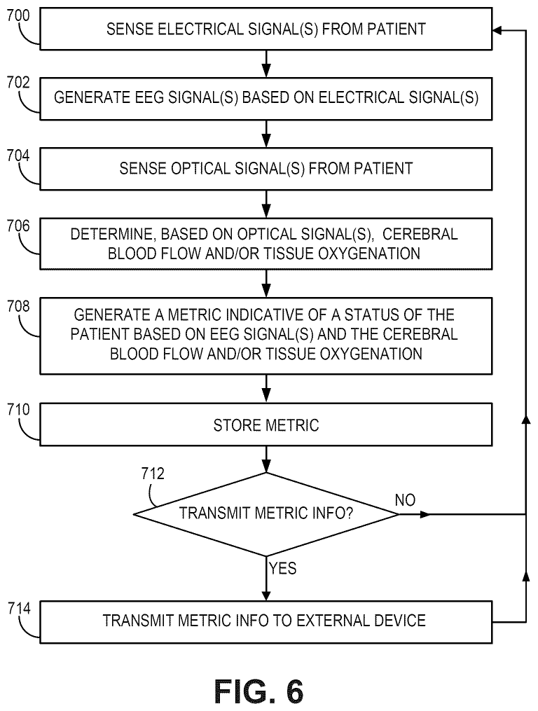

FIG. 6 is a flow diagram of an example technique for generating a stroke status a patient. Lead(s) 10 and processing circuitry 110 will be described as performing the techniques of example FIG. 6, but other components, devices, and systems (e.g., IMD 106 or external device 108) may perform similar functionality in other examples. As shown in the example of FIG. 6, processing circuitry 110 senses, via one or more electrodes 12 of lead 10, electrical signals from the patient (700). Processing circuitry 110 may sense these electrical signals from a sensing vector determined by the electrodes 12 selected for sensing. In this manner, processing circuitry 110 may use different vectors (e.g., different electrode combinations) in order to obtain different electrical information from the patient. Processing circuitry 110 may then generate EEG signal(s) based on the sensed electrical signals (702). Generating EEG signal(s) may include various filtering, amplification, transforms, digitization, or any other conditioning and processing that generates an EEG signal can be analyzed by processing circuitry 110.

Processing circuitry 110 may sense, via one or more optical sensors 16 of lead 10, optical signal(s) from the patient (704). Processing circuitry 110 may determine, based on the optical signal(s), one or more of cerebral blood flow and/or tissue oxygenation of patient (706). Processing circuitry 110 then generates a metric indicative of a status of the patient based on the EEG signal(s) and one or more of the cerebral blood flow and/or tissue oxygenation of the patient (708). For example, the metric may be stroke metric indicative of a stroke status of the patient. In some examples, the metric may be a pressure metric indicative of perfusion pressure of the patient, such as ICP of the patient. For example, processing circuitry 110 may determine perfusion pressure of patient, such as ICP, based on the EEG signal(s) and one or more of the cerebral blood flow and/or tissue oxygenation of the patient. In some examples, processing circuitry may determine a stroke metric based, at least in part, on the determined perfusion pressure of the patient, such as ICP of the patient.

In some examples, when cerebral blood flow is between 35 ml/(100 g min) to 50 ml/(100 g min) and the EEG signal(s) is normal, processing circuitry 110 may indicate a patient may have decreased protein synthesis. In some examples, when cerebral blood flow is between 25 ml/(100 g min) to 35 ml/(100 g min) and the EEG signal(s) indicate a loss of alpha (α) band frequencies, processing circuitry 110 may indicate a patient may have one or more of an anaerobic metabolism or neurotransmitter release. In some examples, when cerebral blood flow is between 18 ml/(100 g min) to 25 ml/(100 g min) and the EEG signal(s) indicate increasing theta (θ) band frequencies, processing circuitry 110 may indicate a patient may have one or more of lactic acidosis or declining ATP. In some examples, when cerebral blood flow is between 12 ml/(100 g min) to 18 ml/(100 g min) and the EEG signal(s) indicate increasing delta (δ) band frequencies, processing circuitry 110 may indicate a patient may have one or more of sodium-potassium pump failure or increased intracellular water content. In some examples, when cerebral blood flow is less than 12 ml/(100 g min)) and the EEG signal(s) indicate signal suppression, processing circuitry 110 may indicate a patient may have one or more of calcium accumulation, anoxic depolarization, or cell death.

In some examples, processing circuitry 110 may determine patient suffered or is suffering an ischemic stroke event based on, at least, determining cerebral blood flow satisfies a low cerebral blood flow threshold and brain tissue oxygenation satisfies a low brain tissue oxygenation threshold. In some examples, processing circuitry 110 may determine patient suffered or is suffering an ischemic stroke event based on, at least, determining cerebral blood flow satisfies a low cerebral blood flow threshold, brain tissue oxygenation satisfies a low brain tissue oxygenation threshold, and cerebral metabolic rate of oxygen satisfies a low cerebral metabolic rate of oxygen threshold.

In some examples, processing circuitry 110 may determine patient suffered or is suffering a hemorrhagic stroke event based on, at least, determining cerebral blood flow satisfies a high cerebral blood flow threshold and brain tissue oxygenation satisfies a high brain tissue oxygenation threshold. In some examples, processing circuitry 110 may determine patient suffered or is suffering a hemorrhagic stroke event based on, at least, determining cerebral blood flow satisfies a high cerebral blood flow threshold, brain tissue oxygenation satisfies a high brain tissue oxygenation threshold, and cerebral metabolic rate of oxygen satisfies a high cerebral metabolic rate of oxygen threshold.

In some examples, processing circuitry 110 may determine a oxy hemoglobin (HbO) signal(s) and/or de-oxy hemoglobin (HbR) signal(s), based on the sensed optical signals and determine a value of one or more of the HbO signal and/or the HbR signals Processing circuitry 110 may determine a baseline value of the HbO and/or HbR, such as an absolute value of the HbO and/or HbR, and determine whether the absolute value of HbO and/or HbR are decreasing over a period of time. In response to the determining the absolute value of HbO and/or HbR are decreasing over a period of time, processing circuitry 110 may determine whether the change in value of the of HbO and/or HbR satisfies a respective HbO change threshold or HbR change threshold. In response to the change in value of the of HbO and/or HbR satisfying a respective HbO change threshold or HbR change threshold, processing circuitry 110 may determine a second order differential of the HbO and/or HbR values and determine whether the second differential values of HbO and/or HbR satisfy a respective second differential HbO threshold or second differential HbR threshold.

In some examples, processing circuitry 110 may determine patient suffered an ischemic stroke event based on determining one or more of the second differential values of HbO and/or HbR satisfy a respective second differential HbO and/or HbR threshold and a second differential of a value of a bandwidth ratio of the EEG signal, such as DAR, satisfies a second differential bandwidth ratio threshold. In some examples, the bandwidth ratio of the EEG signal may be an outcome of spectral power estimates. In some examples, processing circuitry 110 may determine an electrical impedance spectrum over a range of frequencies, such as 10 Hz to 1 MHz, to determine a spectral impedance envelope. In some examples, processing circuitry 110 may further determine patient suffered an ischemic stroke event based on a spectral impedance envelope value satisfying (e.g., greater than) a spectral impedance envelope threshold. In some examples, processing circuitry 110 may determine patient suffered a hemorrhagic stroke event based on determining one or more of the second differential values of HbO and/or HbR does not satisfy a respective second differential HbO and/or HbR threshold and a second differential of a value of a bandwidth ratio of an EEG signal, such as DAR, satisfies a second differential bandwidth ratio threshold. In some examples, processing circuitry 110 may further determine patient suffered a hemorrhagic stroke event based on a spectral impedance envelope value not satisfying (e.g., less than) a spectral impedance envelope threshold.

In some examples, processing circuitry 110 may extract B & M wave(s) from the HbO and/or HbR signals. The B waves may emerge at or near the same time as lower frequencies of the EEG signal, such as delta and theta, oscillate and may correspond to glymphatic flow and/or cerebral blood flow oscillations. Processing circuitry 110 may determine power spectral correlates of the B & M wave(s) and determine a B to M wave ratio (BMR). In some examples, processing circuitry 110 may determine an ischemic stroke occurred or is occurring in response to the BMR satisfying a BMR threshold and a bandwidth ratio of an EEG signal, such as DAR, satisfying an ischemic stroke bandwidth ratio threshold. In some examples, processing circuitry 110 may determine a hemorrhagic stroke occurred or is occurring in response to the BMR satisfying a BMR threshold and a bandwidth ratio of an EEG signal, such as DAR, satisfying a hemorrhagic stroke bandwidth ratio threshold.

In some examples, processing circuitry 110 may store the metrics in memory (710). If processing circuitry 110 has instructions to transmit the metric information to an external device (such as external device 108) (“YES” branch of block 712), processing circuitry 110 may control communication circuitry to transmit the metric information to external device 108 or another computing device (714). For example, instructions to transmit the metric information may include processing circuitry 110 determining or receiving a trigger has been satisfied to send the information such as the stroke metric exceeding a respective threshold that indicates a stroke is occurring or has occurred. For example, processing circuitry 110 may cause the information to be sent to external device 108 or another computing device. In this manner, the external device 108 or another computing device may inform the patient or a clinician that the patient may need assistance or therapeutic intervention. If processing circuitry 110 does not have instructions to transmit the metric information to an external device (such as external device 108) (“NO” branch of block 712), processing circuitry 110 may continue to sense electrical signals from the patient (700).

In some examples, such as system including a one or more IMDs 106 in conjunction with lead(s) 10, upon external device 108 receiving notification of a possible stroke or perfusion pressure being above a pressure threshold due to the trigger being satisfied, external device 108 may send a request to one or more of the IMDs 106 to request signals from the IMDs 106 to make a second stroke determination, such as a stroke confirmation or high perfusion pressure confirmation, which may improve the specificity and/or sensitivity of the stroke determination and/or high perfusion pressure determination.

In some examples, the generated stroke metric satisfying a stroke criteria threshold or pressure metric satisfying a pressure criteria threshold may correspond to a triggering event. In some examples, in response the generated stroke metric satisfying a stroke criteria threshold or pressure metric satisfying a pressure criteria threshold, processing circuitry 110 may send patient data, such as one or more of the EEG signal, the optical signal, cerebral blood flow, and/or tissue oxygenation to another computing device, such as external device 108 or another computing device, for further adjudication. In some examples, the further adjudication may include the external device 108 or another computing device applying an artificial intelligence model (e.g., machine learning, neural networks, etc.) to patient data to determine whether a stroke was detected and/or what type of stroke was detected and/or determine whether perfusion pressure is above a high perfusion pressure threshold. In some examples, processing circuitry 110 may perform the further adjudication itself in response to the triggering event, and apply an artificial intelligence model (e.g., machine learning, neural networks, etc.) to patient data to determine whether a stroke was detected and what type of stroke was detected and/or determine whether perfusion pressure is above a high perfusion pressure threshold.

External device 108 may employ various techniques to determine whether stroke occurred. For example, external device 108 may generate a metric, such as a stroke metric or pressure metric, using one or more different algorithms, such as using machine learning algorithms. In one example, a gradient boosting algorithm can be trained on a data set following feature extraction to generate a classifier algorithm. The classifier can be tuned by paring down features to only those related to the stroke/non-stroke condition. In some examples, external device 108 may apply a classification algorithm for determining that stroke metric or pressure metric can be, for example, an algorithm adapted from the use of artificial intelligence (e.g., machine learning, neural networks, etc.) as applied to patient data to determine what type of stroke was detected. Based on the classification algorithm, block stroke determination is made, which may be binary or probabilistic. If a stroke is detected (e.g., a probabilistic determination exceeds some pre-determined threshold, for example 85% likelihood of stroke), then external device 108 may apply an etiology classifier. In some examples, such an etiology classifier can make a determination (probabilistic or definitive) of the origin of the stroke (e.g., ischemic or hemorrhagic). Such determinations can be made based on collected EEG sensor data in conjunction with physiological parameters based on collected optical signals, such as cerebral blood flow and/or tissue oxygenation. In some examples, such determinations may further include additional physiological parameters or patient data, such as posture or activity data obtained from an accelerometer signal and/or inferred based on a clock.

The accuracy of any classifier can be improved by training the algorithm on larger sets of data corresponding to stroke and non-stroke EEG readings. Additionally, other physiological parameters can be added to the classifier model (e.g., activity and/or posture data as determined using an accelerometer, timing data, particular heart rhythms, gender, age, medical history, etc.). In some examples, a classifier can be used to discriminate between ischemic and hemorrhagic strokes. Such discrimination can be particularly useful as the interventions may differ. For example, an ischemic stroke may be treated using thrombectomy, while a hemorrhagic stroke may be treated using surgery or another suitable technique.

As described herein the stroke metric can be, for example, a binary output of stroke condition/non-stroke condition, a probabilistic indication of stroke likelihood, or other output relating to the patient's condition and likelihood of having suffered a stroke. This stroke metric can be calculated using a classifier model as described elsewhere herein.

When processing circuitry 110 transmits the stroke metric to an external device, the external device may be associated with emergency services in some examples. In some examples, the external device may include global position system (GPS) capability or other location detection technology (e.g., WiFi triangulation) such that the external device can identify, store, and/or communicate the geographic location at which the stroke metric occurred. The external device may then transmit the location information and/or stroke metric to another device or system via cell phone tower, satellite, or other technology. The other system may be an emergency service such as 911 or other medical service. If the technique of FIG. 6 is performed in an ambulance, for example, a device carried by the ambulance or technician may receive the metric and output information or instructions to an emergency medical technician (EMT) or other personnel in the rear of the ambulance and/or to the ambulance driver. In some embodiments, the display to the ambulance driver can include navigational information such as a map and instructions to take the patient to a particular hospital or facility with a stroke center.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A lead includes a plurality of electrodes configured to sense electrical signals from a patient; one or more light emitting diodes (LEDs); and one or more optical sensors configured to sense optical signals, wherein the LEDs, the optical sensors and the electrodes are respectively aligned in measuring pairs, the measuring pairs including: one of the LEDs or one of the optical sensors, and one of the plurality of electrodes.

Example 2: The lead of example 1, wherein the measuring pairs include one or more first measuring pairs and one or more second measuring pairs, the first measuring pair including one of the LEDs and one of the plurality of electrodes, the second measuring pair comprising one of the optical sensors and one of the plurality of electrodes.

Example 3: The lead of example 2, wherein the first measuring pairs and the second measuring pairs are aligned alternatively in an axial direction of the lead.

Example 4: The lead of any of examples 1-3, wherein each of the measuring pairs are respectively separated from an adjacent measuring pair of the measuring pairs by at least a predetermined distance.

Example 5: The lead of any of examples 1 through 4, wherein when the lead is implanted on a head of the patient, at least one measuring pair of the measuring pairs is positioned on a front hemisphere of the head of the patient and at least one measuring pair of the measuring pairs is positioned on a back hemisphere of the head of the patient.

Example 6: The lead of any of examples 4 and 5, wherein the predetermined distance is 4 centimeters.

Example 7: The lead of any of examples 1-6 further comprising a controller electrically coupled to the measuring pairs, the controller comprising a power source, a communication interface, processing circuitry, and sensing circuitry.

Example 8: The lead of examples 7, wherein the power source is a battery.

Example 9: The lead of any of examples 1-6 further comprising one or more contacts configured to electrically couple the plurality of electrodes, the LEDs, and the optical sensors to a controller.

Example 10: The lead of any of examples 1-9, wherein the LEDs include one or more near-infrared (NIR) LEDs.

Example 11: The lead of any of examples 1-10, wherein the plurality of electrodes are configured to sense the electrical signals during a same period of time as the one or more optical sensors are configured to sense optical signals.

Example 12: The lead of any of examples 1-11, wherein the one or more optical sensors are photodiodes.

Example 13: The lead of any of examples 1-12, wherein the lead is a subcutaneous lead.

Example 14: A system includes a memory; a lead includes a plurality of electrodes; a plurality of near-infrared (NIR) light emitting diodes (LED); and a plurality of optical sensors configured to sense optical signals; processing circuitry configured to: sense electrical signals from a patient during a period of time; and generate, based on the electrical signals, one or more electroencephalography (EEG) signals; sense, via at least one of the optical sensors and using at least one of the LEDs, optical signals during the period of time; determine, based on the optical signals, one or more of cerebral blood flow or blood oxygenation; generate a metric indicative of a status of the patient based on the one or more EEG signals and the one or more of cerebral blood flow or blood oxygenation; and store the metric in the memory.

Example 15: The system of example 14, wherein the metric is a stroke metric indicative of a stroke status of the patient.

Example 16: The system of any of examples 14 and 15, wherein the metric is a pressure metric indicative of an intracranial pressure of the patient.

Example 17: The system of any of examples 14-16, wherein the processing circuitry is further configured to: in response to one or more of visual stimuli or audio stimuli being applied to the patient, sense, via at least one of the plurality of electrodes, one or more of visual evoked potential (VEP) or auditory evoked potential (AEP); and determine whether one or more of the VEP or the AEP satisfies a stroke threshold.

Example 18: The system of example 17, wherein the processing circuitry is further configured to: in response to determining one or more of the VEP or the AEP satisfies a stroke threshold, determine a stroke occurred in the patient.

Example 19: The system of any of examples 17 and 18, wherein the processing circuitry is further configured to: perform polyspectral analysis on a second EEG signal, sensed via one or more of the electrodes, the second EEG signal being from a timepoint before the visual stimuli or audio stimuli are applied to the patient to a timepoint after the visual stimuli or audio stimuli are applied to the patient.

Example 20: The system of example 19, wherein the processing circuitry is further configured to: in response to the polyspectral analysis on the second EEG signal satisfying a stroke threshold, determine a stroke occurred in the patient.

Example 21: The system of any of examples 14 through 20, further includes receive the one or more physiological parameters of the patient from the IMD; and generate the metric indicative of a status of the patient based on the one or more EEG signals, the one or more physiological parameters of the patient from the IMD, and the one or more of cerebral blood flow or blood oxygenation.

Example 22: The system of example 21, wherein the metric is a stroke metric indicative of a stroke status of the patient.

Example 23: The system of any of examples 14-22, wherein the lead is a subcutaneous lead.

Various examples have been described. These and other examples are within the scope of the following claims.

What is claimed is:

1. A lead comprising:
a plurality of electrodes configured to sense electrical signals from a patient;
one or more light emitting diodes (LEDs); and
one or more optical sensors configured to sense optical signals,
wherein the LEDs, the optical sensors and the electrodes are respectively aligned in measuring pairs, the measuring pairs including:
one of the LEDs or one of the optical sensors, and
one of the plurality of electrodes.

2. The lead of claim 1, wherein the measuring pairs include one or more first measuring pairs and one or more second measuring pairs, the first measuring pair including one of the LEDs and one of the plurality of electrodes, the second measuring pair comprising one of the optical sensors and one of the plurality of electrodes.

3. The lead of claim 2, wherein the first measuring pairs and the second measuring pairs are aligned alternatively in an axial direction of the lead.

4. The lead of claim 1, wherein each of the measuring pairs are respectively separated from an adjacent measuring pair of the measuring pairs by at least a predetermined distance.

5. The lead of claim 1, wherein when the lead is implanted on a head of the patient, at least one measuring pair of the measuring pairs is positioned on a front hemisphere of the head of the patient and at least one measuring pair of the measuring pairs is positioned on a back hemisphere of the head of the patient.

6. The lead of claim 4, wherein the predetermined distance is 4 centimeters.

7. The lead of claim 1 further comprising a controller electrically coupled to the measuring pairs, the controller comprising a power source, a communication interface, processing circuitry, and sensing circuitry.

8. The lead of claim 1 further comprising one or more contacts configured to electrically couple the plurality of electrodes, the LEDs, and the optical sensors to a controller.

9. The lead of claim 1, wherein the LEDs include one or more near-infrared (NIR) LEDs.

10. The lead of claim 1, wherein the plurality of electrodes are configured to sense the electrical signals during a same period of time as the one or more optical sensors are configured to sense optical signals.

11. The lead of claim 1, wherein the lead is a subcutaneous lead.

12. A system comprising:
a memory;
a lead comprising:
a plurality of electrodes;
a plurality of near-infrared (NIR) light emitting diodes (LED); and
a plurality of optical sensors configured to sense optical signals;
processing circuitry configured to:
sense electrical signals from a patient during a period of time; and
generate, based on the electrical signals, one or more electroencephalography (EEG) signals;
sense, via at least one of the optical sensors and using at least one of the LEDs, optical signals during the period of time;
determine, based on the optical signals, one or more of cerebral blood flow or blood oxygenation;
generate a metric indicative of a status of the patient based on the one or more EEG signals and the one or more of cerebral blood flow or blood oxygenation; and
store the metric in the memory.

13. The system of claim 12, wherein the metric is a stroke metric indicative of a stroke status of the patient.

14. The system of claim 12, wherein the metric is a pressure metric indicative of an intracranial pressure of the patient.

15. The system of claim 12, wherein the processing circuitry is further configured to:
in response to one or more of visual stimuli or audio stimuli being applied to the patient, sense, via at least one of the plurality of electrodes, one or more of visual evoked potential (VEP) or auditory evoked potential (AEP); and
determine whether one or more of the VEP or the AEP satisfies a stroke threshold.

16. The system of claim 15, wherein the processing circuitry is further configured to:
in response to determining one or more of the VEP or the AEP satisfies a stroke threshold, determine a stroke occurred in the patient.

17. The system of claim 15, wherein the processing circuitry is further configured to:
perform polyspectral analysis on a second EEG signal, sensed via one or more of the electrodes, the second EEG signal being from a timepoint before the visual stimuli or audio stimuli are applied to the patient to a timepoint after the visual stimuli or audio stimuli are applied to the patient.

18. The system of claim 17, wherein the processing circuitry is further configured to:
in response to the polyspectral analysis on the second EEG signal satisfying a stroke threshold, determine a stroke occurred in the patient.

19. The system of claim 12, further comprising an implantable medical device (IMD) configured to sense one or more physiological parameters of the patient, the IMD being separate from the lead,
wherein the processing circuitry is further configured to:
receive the one or more physiological parameters of the patient from the IMD; and
generate the metric indicative of a status of the patient based on the one or more EEG signals, the one or more physiological parameters of the patient from the IMD, and the one or more of cerebral blood flow or blood oxygenation.

20. The system of claim 19, wherein the metric is a stroke metric indicative of a stroke status of the patient.
